(19) United States
(12) Patent Application Publication
(10) Pub. No.: US 2024/0207049 A1
(43) Pub. Date: Jun. 27, 2024

(54) TRANSCATHETER DEVICE FOR TREATING TRICUSPID VALVE REGURGITATION

(71) Applicants: TAU MEDICAL INC., Busan (KR); TAU MEDICAL INC., Centreville, VA (US)

(72) Inventor: June-Hong Kim, Busan (KR)

(21) Appl. No.: 18/392,344

(22) Filed: Dec. 21, 2023

Related U.S. Application Data
(60) Provisional application No. 63/434,355, filed on Dec. 21, 2022.

(51) Int. Cl. A61F 2/24; A61F 2/48

(52) U.S. Cl. CPC A61F 2/246; A61F 2/2466; A61F 2/484; A61F 2250/0003; A61M 25/1002

(57) ABSTRACT

A transcatheter device for treating the tricuspid valve regurgitation. The transcatheter device comprises a main shaft, a proximal portion, a distal tail, and a spacer body mounted on the main shaft and located between the proximal portion and the distal tail. This transcatheter device could be used for treating tricuspid valve regurgitation in a patient's heart. All or part of the transcatheter device is supported by the main shaft. The spacer body is mounted on the shaft, which travels through the spacer body. Also disclosed are a coaptation assembly that comprises the transcatheter device, and a method of treating tricuspid valve regurgitation using the transcatheter device.

The present application claims the benefit of U.S. Provisional Application No. 63/434,355, filed on Dec. 21, 2022; all of which is incorporated herein by reference in its entirety.

### TECHNICAL FIELD

The present invention relates to the treatment of tricuspid valve regurgitation by using a transcatheter device.

### BACKGROUND

Heart valve regurgitation (leakage through a heart valve) occurs when a heart valve fails to close properly. One example is tricuspid valve regurgitation, which is typically caused by changes in the geometric configurations of the right ventricle, papillary muscles, and tricuspid valve annulus. These geometric alterations result in incomplete leaflet coaptation during ventricular systole, thereby producing regurgitation. In the past, repairing heart valves required open-heart surgery with cardiopulmonary bypass. In recent years, a variety of catheter-based techniques for valve repair are being introduced. These catheter-based procedures do not require opening the chest or the use of cardiopulmonary bypass. There is need for further advancement in catheter-based treatments for tricuspid valve regurgitation.

### SUMMARY

TRANSCATHETER DEVICE: In one aspect, this invention is a transcatheter device comprising a main shaft, a proximal portion, a distal tail, and a spacer body (inflatable balloon) mounted on the main shaft and located between the proximal portion and the distal tail. This transcatheter device could be used for treating tricuspid valve regurgitation in a patient's heart. All or part of the transcatheter device is supported by the main shaft. The spacer body is mounted on the shaft, which travels through the spacer body. The proximal portion of the transcatheter device encompasses a proximal segment of the main shaft. This could be expressed alternatively as the proximal segment of the main shaft comprising the proximal portion of the transcatheter device. The distal tail of the transcatheter device encompasses a distal segment of the main shaft. This could be expressed alternatively as the distal segment of the main shaft comprising the distal tail of the transcatheter device.

The main shaft comprises a lumen and opening(s) for admitting a guidewire therethrough. There could be an opening for the lumen at the distal tip of the main shaft (in the distal tail part). There could also be a proximal opening located at the proximal portion of the transcatheter device. In embodiments where the transcatheter device comprises an intravascular anchor, this proximal opening for the lumen could be located between the spacer body and the intravascular anchor. For example, the opening could be located at the proximal end of the main shaft where it joins the intravascular anchor.

The total length of the main shaft could be in the range of 50-175 cm long. The main shaft may be constructed in any suitable way. For example, it could be made of a metal wire core (e.g. stainless steel or nitinol alloy), which is then covered the a polymer material. For example, the metal wire core could be covered with thermoplastic polyurethane braiding or polytetrafluoroethylene (PTFE) coating. The metal wire core could extend through the full length of the main shaft. However, in some embodiments, the metal wire core terminates before reaching the tip of the distal tail (or distal tip of main shaft). For example, the metal wire core could terminate at a location that is within 0.5-4 cm of the distal tip.

Distal Tail. The distal tail may have any suitable length to provide sufficient anchoring within the pulmonary artery. In some embodiments, the length of the distal tail is 10-40 cm long; and in some cases, 15-30 cm long. The distal tail could have a pigtail or rounded tip to blunt the tip and reduce trauma as it travels into pulmonary artery. In some embodiments, the distal tail has one or more bends. The bend(s) could have an inner angle in the range of 80-140°. The bends(s) could be located at any suitable location on the distal tail. In some embodiments, there is a bend located at a distance of 0.25-3.5 cm from the spacer body.

The distal tail may have a non-constant diameter over its length. In some embodiments, the distal tail comprises a proximal segment and a distal segment. The proximal segment may encompass 10-60% of the total length of the distal tail. The distal segment could have a thinner diameter than the proximal segment. There may be various reasons for this difference, such as the proximal segment having more or thicker sheathing or covering than the distal segment. The distal segment could be more flexible than the proximal segment of the distal tail. In some embodiments, the distal tail does not comprise any coil, loop, or stent.

The distal tail could be designed to have a streamlined shape. In some embodiments, the distal tail is a thin elongate cylinder shape (with or without a lumen) with no protruding features, such as hooks, wires, rings, ridges, etc. This may be useful in preventing thrombus formation or erosion of the distal tail into the wall of the pulmonary artery.

In some embodiments, the distal segment is more flexible than the proximal segment. In some cases, the proximal segment comprises a metal braiding, whereas the distal segment does not. The distal segment could comprise a polymer material that is softer than the proximal segment. The distal segment could have a smaller diameter than the proximal segment. In some cases, the length of the distal segment is shorter than the length of the proximal segment. The length of the distal segment could be 2-7 cm long. The length of the proximal segment could be 7-15 cm long. In some cases, the proximal segment constitutes 35-65% of the total length of the distal tail.

The proximal segments could have a different size than the distal segment. In some cases, the distal segment has a smaller diameter than the proximal segment. In some cases, the diameter of the distal segment is 45-85% of the diameter size of the proximal segment. For example, the distal segment could have a diameter of 2-5 mm, whereas the proximal segment could have a diameter of 3-6 mm.

In some cases, the distal tail further comprises a middle segment between the proximal segment and the distal segment. The middle segment is more flexible than the proximal segment but is stiffer than the distal segment. In some cases, the length of the middle segment is shorter than the length of the proximal segment. For example, the length of the middle segment could be 2-7 cm long.

Spacer Body or Balloon. The spacer body is mounted on the main shaft. The spacer body is made to have dimensions or shape suitable for providing a coaptation surface for leaflets of the tricuspid valve. For example, the shape of the spacer body may have a particular design. In some embodiments, the spacer body has a linear shape (e.g. ovoid, cylindrical with tapered or conical ends, etc.). In some embodiments, the spacer body has a non-linear shape (e.g. curved or boot-shaped). In a non-linear shaped spacer body, the spacer body could comprise a bend having an inner angle in the range of 80-140°.

Another design parameter is the length of the spacer body. For example, the spacer body could be 4-13 cm long; and in some cases, 5-9 cm. In cases where the spacer body has a non-linear shape, this length is represented by the travel distance along the longitudinal axis of the spacer body. The width of the spacer body can be measured on a transverse cross-section plane that is orthogonal to the longitudinal axis. In some embodiments, the widest width of the spacer body on this transverse cross-section plane is in the range of 0.5-3.5 cm; and in some cases, in the range of 0.5-2.5 cm. The spacer body may have a relaxed contracted configuration and an elongated configuration. In this situation, the measurements above for the spacer body are made in the relaxed configuration. In some embodiments, the width of the spacer body on the widest axis is greater than the width of the spacer body on its cross-axis on the transverse plane (i.e. non-circular or asymmetric cross-section).

The spacer body can have any suitable structure, such as balloon (e.g. fluid, foam, or air-filled), basket, mesh, struts (e.g. like a stent), framework, skeleton, scaffolding, etc. If needed, a surface for the spacer body may be provided in any suitable manner, such as a skin, shell, casing, or membrane. The spacer body may be made of any suitable material, such as plastics, metals, or combinations thereof. The spacer body could have one or more openings to allow the flow of blood therethrough. There may be a gap between the spacer body (at one of its ends) and the main shaft to allow the flow of blood therethrough. These openings or gaps allows blood to flow easily through the spacer body, which may be useful for preventing thrombus formation.

In some embodiments, the spacer body comprises one or more side appendages. These may be located on a lateral side of the spacer body. The side appendage can be any type of thin and flexible structure that enhances the function of the spacer body as a barrier against the flow of blood across gaps in the tricuspid valve leaflets. Examples of side appendages include wings, flaps, shrouds, drapes, skirts, free edges, tags, etc. The side appendage has a widened configuration (for ventricular systole) and a narrowed configuration (for ventricular diastole).

The widened configuration for the side appendage is induced by the direction of blood flow and could be performed in any suitable manner, such as spreading out, extending out, enlarging, distending, folding out, opening, etc. The narrowed configuration for the side appendage is induced by the other direction of blood flow and could be performed in any suitable manner, such as folding in, retracting, collapsing, shrinking, closing, etc.

The side appendage should be sufficiently wide to reduce gaps between the tricuspid valve leaflets or help stabilize the spacer body across the tricuspid valve. In some embodiments, the width of the side appendage is 0.3-5.0 cm; and in some cases, 0.5-3.5 cm. The width is measured as the widest distance for the side appendage from spacer body in a direction that is orthogonal to the transverse axis of the spacer body.

The length of the side appendage may be shorter than the length of the spacer body. In some embodiments, the length of the side appendage is 2-9 cm; and in some cases, 4-7 cm. The length is the longest length as measured along the longitudinal axis of the spacer body.

The side appendage should be sufficiently thin to be flexibly responsive to blood flow across the tricuspid valve. In some embodiments, the side appendage has a thickness of 0.2-10 mm; and in some cases, 0.3-6 mm. The thickness is measured along a transverse axis of the spacer body that is orthogonal to the side appendage and the longitudinal axis of the spacer body.

The side appendage can have any suitable shape. In some embodiments, the side appendage has a non-flat shape with a three-dimensional curvature that gives the side appendage an inner side (concave) and an outer side (convex). Having this non-flat shape may be useful for improving the response to blood flow across the tricuspid valve.

Proximal Portion. The proximal portion of the transcatheter device comprises the proximal segment of the main shaft. The proximal segment could be a proximal continuation of the main shaft. The proximal portion of the transcatheter device could have any suitable length to provide intravascular access or sufficient anchoring within the vena cava. In some embodiments, the total length of the proximal portion is in the range of 10-60 cm long. In embodiments where the proximal portion includes an intravascular anchor, this measurement includes the length of the intravascular anchor. In situations where the intravascular anchor does not have a linear shape (e.g. coil), this means the length as measured along the longitudinal axis.

In some embodiments, the proximal segment of the main shaft has one or more bends. The bend(s) could have an inner angle in the range of 80-140°. The bends(s) could be located at any suitable location on the proximal segment of the main shaft. In some embodiments, there is a bend located at a distance of 0.25-5.5 cm from the spacer body. The proximal segment could also have a curved portion (wider than a bend). In some embodiments, the proximal segment has two separate bends and a curved portion between the two bends. The length of the proximal segment could be in the range of 3-15 cm long.

Intravascular Anchor. In some embodiments, the proximal portion comprises an intravascular anchor. Examples of intravascular anchors include spiral coil and expandable stent. In some embodiments, the intravascular anchor is a spiral coil. The spiral coil could have at least two spirals. The intravascular anchor could have any suitable width for anchoring in the vena cava. In some embodiments, the widest width of the intravascular anchor is in the range of 2-7 cm wide. The length of the intravascular anchor could be in the range of 4-11 cm long (as measured straight on its longitudinal axis). In situations where the intravascular anchor does not have a linear shape (e.g. coil), this means the length as measured along the longitudinal axis. In situations where the intravascular anchor has flexible configurations (e.g. as in a helical coil), this length is measured in its naturally coiled configuration. In an alternate embodiment of this invention, the transcatheter device comprises either the intravascular anchor or the distal tail, but not both.

Radiopaque Markers. The transcatheter device may have one or more radiopaque markers that are visible under x-ray imaging (e.g. x-ray fluoroscopy). In some embodiments of the transcatheter device, there is a first radiopaque marker that is located on the proximal segment of the main shaft (proximal to the spacer body), and a second radiopaque that is located on the distal tail (distal to the spacer body). The first radiopaque marker could be located within 2 cm of the proximal end of the spacer body. The second radiopaque marker could be located within 2 cm of the distal end of the spacer body.

COAPTATION ASSEMBLY: In another aspect, this invention is a coaptation assembly for treating tricuspid valve regurgitation. The assembly comprises a transcatheter device of the invention. The assembly further comprises a guidewire traveling through the lumen of the main shaft. In some embodiments, the assembly further comprises a moveable delivery sheath that can cover the spacer body or intravascular anchor. The sheath could be advanced to cover the spacer body or intravascular anchor. Or the spacer body could be retracted to uncover the spacer body or intravascular anchor. In some embodiments, the assembly further comprises a deployment catheter. The deployment catheter is sufficiently long to deploy the transcatheter device in the patient's heart. For example, the deployment catheter could be 50-150 cm long.

COAPTATION KIT: In another aspect, this invention is a coaptation kit for treating tricuspid valve regurgitation. The kit comprises a transcatheter device of the invention, a deployment catheter, a delivery sheath, and a guidewire. These components could be assembled or used in the manner described herein.

METHOD OF TREATMENT: In another aspect, this invention is a method of treating a defective tricuspid valve in a patient using a transcatheter device of this invention. The transcatheter device is implanted with the distal tail within the pulmonary artery and the spacer body across the tricuspid valve. The transcatheter device is inserted into an entry vein, such as the femoral, subclavian, or jugular vein. The transcatheter device is advanced further into the vena cava (inferior or superior). The transcatheter device is advanced through a right atrium of the heart, across the tricuspid valve, and into a right ventricle of the heart. The transcatheter device is further advanced towards the pulmonary artery. The distal tail is advanced into the pulmonary artery. This could be the left-side or right-side pulmonary artery.

The distal tail works to help anchor the transcatheter device. As such, the distal tail may extend into the pulmonary artery of sufficient distance to perform this function. In some embodiments, the distal tail extends for a distance of at least 10 cm into the pulmonary artery; and in some cases, at least 15 cm. In some embodiments, the distal tail is advanced past a first branching point of the pulmonary artery; in some cases, past a second branching point of the pulmonary artery; and in some cases past a third branching point of the pulmonary artery. Proper positioning of the distal tail could be confirmed by having a radiopaque marker and x-ray imaging to see the radiopaque marker. In some embodiments, the distal tail is not embedded within heart tissue.

The spacer body should be properly positioned between leaflets of the tricuspid valve. This proper placement could be confirmed by external imaging such as x-ray or echocardiogram. In some embodiments, the spacer body is positioned to abut against a supraventricular crest of the heart. This abutment against the supraventricular crest could occur at a location within the distal half of the spacer body. The tricuspid valve has a tricuspid annulus and there is an annular plane defined for the tricuspid annulus. This annular plane is along an x-axis of the tricuspid annulus and orthogonal to a Y-axis of the tricuspid annulus. In some embodiments, the spacer body is positioned at an oblique angle)(<90° relative to the annular plane. This oblique angle could be in the range of 15-75°.

In embodiments where the spacer body comprises a side appendage, the method could further comprise widening the side appendage during ventricular systole and narrowing the side appendage during ventricular diastole. In the widened configuration, the side appendage may be positioned between leaflets of the tricuspid valve and obstruct gaps that exist therein. In situations where the side appendage has a non-flat shape, the inner side (concave) is oriented to face towards the right ventricle.

In embodiments where the transcatheter device further comprises an intravascular anchor at its proximal portion, this intravascular anchor is lodged in the vena cava (inferior or superior). The transcatheter device could be implanted using a guidewire. The guidewire is inserted into an entry vein, such as the femoral vein and advanced further into the vena cava (inferior or superior). The guidewire is advanced through the right atrium of the heart, across the tricuspid valve, and into the right ventricle of the heart. The guidewire is further advanced towards the pulmonary artery. The guidewire is inserted into a guidewire lumen of the transcatheter device and the transcatheter device is advanced over this guidewire.

Deployment. The transcatheter device could be deployed using a delivery sheath and deployment catheter. During insertion, the delivery sheath could be moved to cover the spacer body, and for relevant embodiments, cover the intravascular anchor. During deployment the delivery sheath is retracted backwards. Retraction of the delivery sheath and unsheathing components of the transcatheter device could be part of the implantation process. In embodiments where the spacer body is self-expanding, this unsheathing could allow the spacer body to self-expand outward to provide a wider coaptation surface. In embodiments where the transcatheter device comprises an intravascular anchor having an expandable configuration, unsheathing allows the anchor to expand outward to lodge within the vena cava.

In some embodiments, this deployment assembly is not disassembled immediately after the procedure is completed. The clinician may wish to implement a short trial period to confirm the effectiveness of the device. For this short trial period, one or more components of the delivery assembly (deployment catheter, delivery sheath, or guidewire) could be retained inside the patient's body, along with the transcatheter device. During the short trial period, the tricuspid valve function is monitored (e.g. by echocardiogram). If the transcatheter device shows effectiveness during this trial period, the deployment assembly is removed, but retaining the transcatheter device in place. If the trial period shows ineffective results, having the deployment assembly still-in-place allows easy removal of the transcatheter device. The trial period could be any suitable short duration. For example, the trial period could be a duration that is within the range of 12-48 hours post-insertion.

Retrieval. After being implanted, the transcatheter device could be removed if needed. This can be done by grasping the intravascular anchor (e.g. spiral coil at its proximal tip) and pulling out the transcatheter device for removal from the patient's body. For example, this could be performed by inserting a snare catheter through an entry vein, advancing the snare catheter towards the spiral coil, grasping the spiral coil, withdrawing the snare catheter, and pulling out the transcatheter device from the entry vein.

### BRIEF DESCRIPTION OF THE DRAWINGS

FIG. 1A shows an example of a transcatheter device for treating tricuspid regurgitation.

FIG. 1B shows a side view of the transcatheter device.

FIG. 1C shows a cross-section view of the transcatheter device.

FIG. 1D shows an expanded view of the valve of the device.

FIG. 1E shows an expanded view of the balloon of the transcatheter device.

FIG. 1F shows the main shaft of the device in the proximal to the distal segment of the transcatheter device.

FIGS. 2A-2C show the detailed views of the distal segment of the transcatheter device.

FIGS. 3A-3D show the detailed views of the middle segment of the transcatheter device.

FIGS. 4A-4E show the detailed views of the proximal segment of the transcatheter device.

FIGS. 5A-5C show the detailed views of the control segment of the transcatheter device.

FIG. 6 shows the transcatheter device placed in the heart.

FIGS. 7A-7C show the procedures of the transcatheter device.

### DETAILED DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

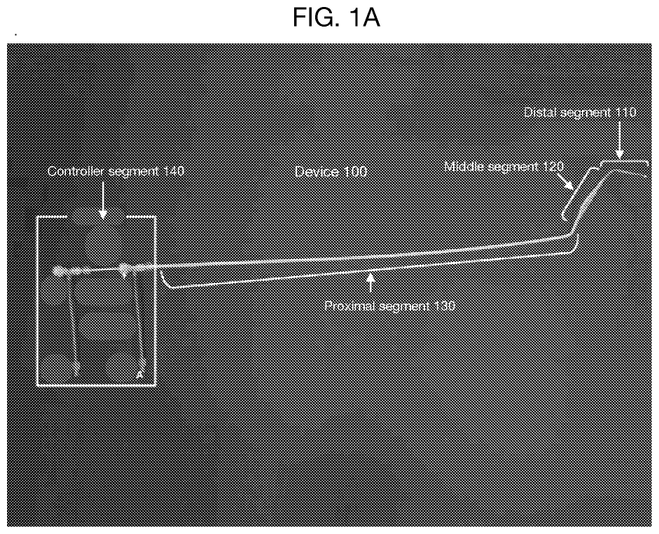

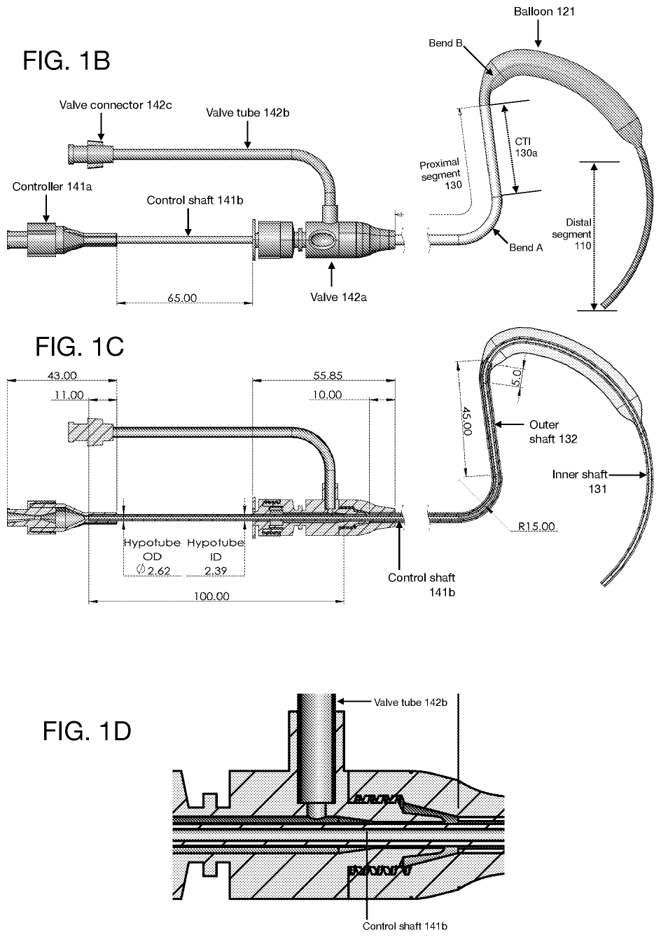

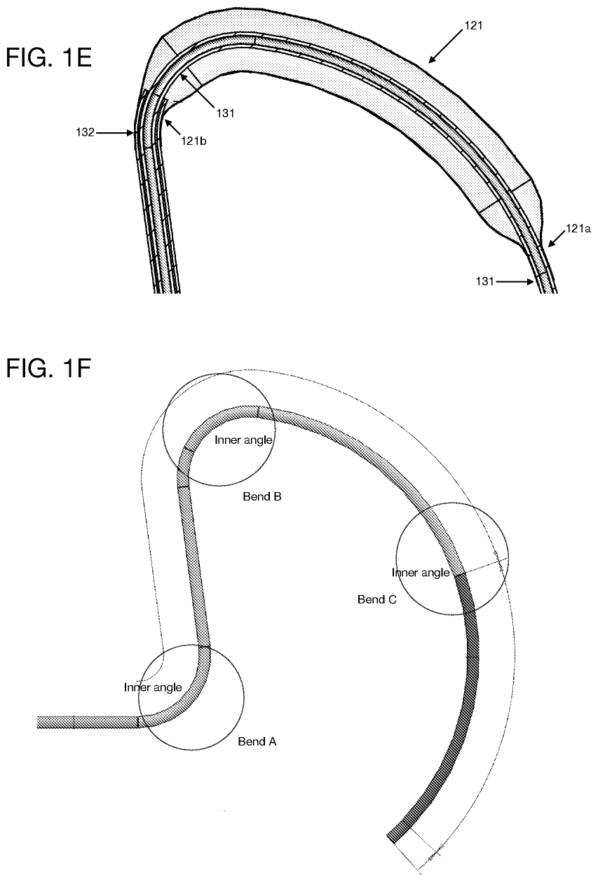

In one embodiment, FIG. 1A shows the transcatheter device 100 comprising a distal segment 110, a middle segment 120, a proximal segment 130, and a controller segment 140. This device 100 could be used for treating tricuspid valve regurgitation in a patient's heart.

In another embodiment, FIG. 1B shows a side view of the device 100, while FIG. 1C shows the cross-section view of the side view of the device 100. The device 100 comprises the distal segment 110, the middle segment 120, the proximal segment 130, and the control segment 140. The proximal segment 130 further comprises a CTI portion 131. The control segment 140 further comprises a control shaft 141.

### Inner and Outer Shafts

In one embodiment, the device 100 further comprises an inner shaft 131 and an outer shaft 132 as shown in FIG. 1C. The inner shaft 131 has a distal end and a proximal end. And the outer shaft 132 has a distal end and a proximal end. The inner shaft 131 runs from the proximal segment 130, the middle segment 120, and the distal segment 110. The outer shaft 132 comprises a lumen for insertion of the inner shaft 131. The inner shaft 131 also comprises a lumen for insertion of a guidewire.

### CTI Portion

In one embodiment, the proximal segment 130 comprises a CTI portion defined in the distal portion between the Bend A and Bend B as shown in FIG. 1B. The length of the CTI portion can be varied depending on the patient's anatomical structure. The CTI portion may have a length of 45-65 mm.

### Pre-defined Natural Bends

In one embodiment, the device 100 may have pre-defined natural bends on the proximal segment 130, the middle segment 120, and the distal segment 110. For example, as shown in FIG. 1E, there is Bend A in the proximal segment 130 and the inner angle of the Bend A has a range of 80-110 degree. The middle segment 120 also has the bend B having an inner angle range of 80-110 degree. Finally, the distal segment 110 has the bend C having an inner angle range of 150-180 degree. Each bends is configured to act as anchor when the device 100 is placed within the IVC, tricuspid valve, and the pulmonary artery. In another embodiment, the portions between each bend may have a pre-defined natural curve as shown in FIG. 1E.

### Inflatable Balloon

In one embodiment, the inflatable balloon 121 comprises a distal end 121a and a proximal end 121b. As shown in FIG. 1D, the distal end 121a of the balloon 121 is securely mounted on the inner shaft 131. The proximal end 121b of the balloon 121 is also securely mounted on the distal portion of the outer shaft 132.

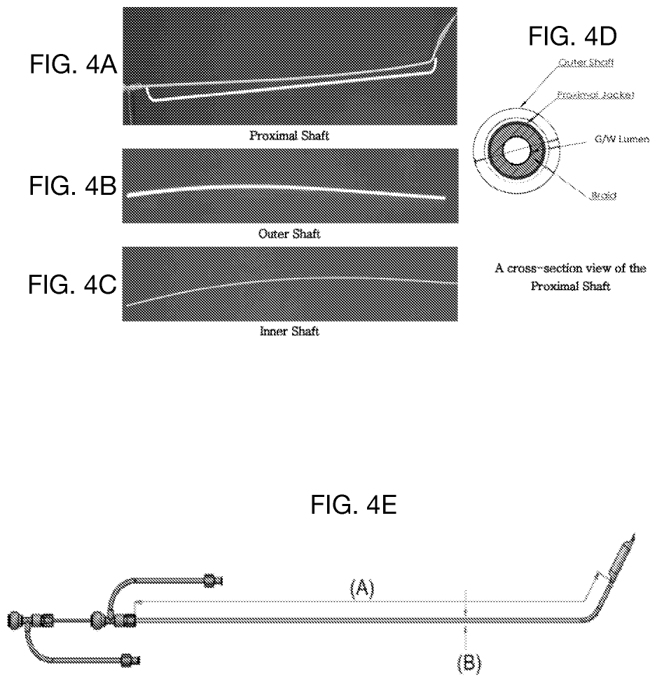

As shown in FIGS. 1D and 4D, there is an inflation lumen created between the inner shaft 131 and outer shaft 132. The lumen is configured for injection of a physiological saline into the balloon 121 for inflation or deflation. The inflation lumen is further connected to the valve tube 142b of the valve 142a as shown in FIGS. 1B and 1C for a fluid communication with a syringe (not shown) through the valve connector 142c as shown in FIGS. 1B and 1C.

In one embodiment, the control shaft 141b is securely attached to the proximal end of the inner shaft 131 in such a way the control shaft 141 is configured to push or pull the inner shaft 131. Also, the control shaft 141 is configured to rotate the inner shaft 131. Since the distal end 121a of the balloon 121 is securely mounted on the inner shaft 131 as shown in FIG. 1D, the balloon shape can be changed by pulling or pushing the control shaft 141b of the controller 141 by the operator.

In one embodiment, when the control shaft 141 is pushed, the balloon can be stretched along the Bend B point. Alternatively, when the control shaft 141 is pulled, the balloon can be further bent inwardly. In another embodiment, when the control shaft 141 is rotated, the balloon is rotated accordingly. Therefore, the operator can accurately control the balloon 121 to place it within the tricuspid valve to reduce the regurgitation by changing the balloon's size, shape, and position while the device 100 is in place.

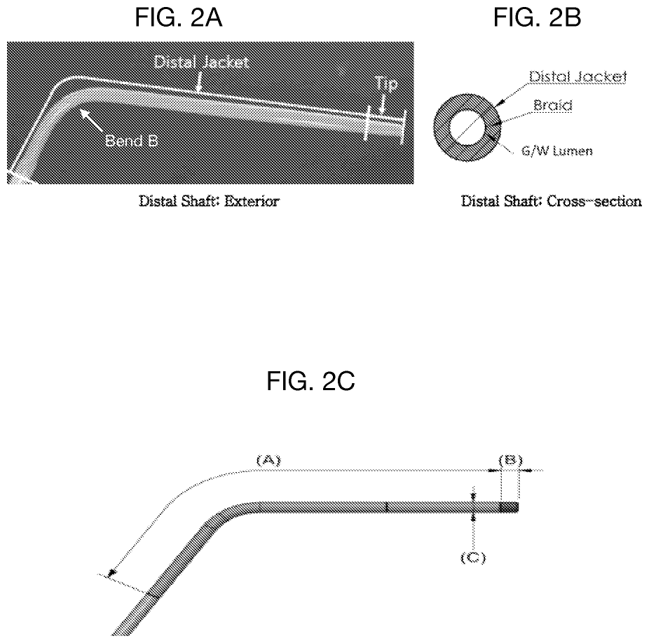

In another embodiment, FIGS. 2A and 2B show the details of the distal shaft (or segment) 110. The distal shaft 110 comprises a pre-determined natural Bend B as shown in FIG. 2A. The bend B has an inner angle in the range of 80-120 degree. FIG. 2B shows a cross-section view of the distal shaft 110. The distal shaft 110 has a lumen for insertion of a guidewire. The distal shaft 110 is configured to be inserted into the pulmonary artery. The distal shaft 110 has at least one lumen with a UV impermeable material that allows the insertion of a guidewire with flexibility. The distal jacket is made of softer Pebax material than the proximal shaft (or segment) 130 to allow easier wrapping of the superventricular crest towards the pulmonary artery during the insertion of inner shaft 131.

The distal shaft 110 further comprises a tip at its distal end. The tip is made of soft material to prevent damage even upon a contact with a blood vessel or surrounding tissue during the insertion of device along the guidewire lumen. The braid in the distal shaft 110 is for the control of the shaft strength and flexibility.

FIG. 2C shows the pre-defined dimensions of the distal shaft 110 having various lengths and diameters depending on the patient's anatomical structure. (Table A).

TABLE A

| | | (A + B) | (A) | | |
|---|---|---|---|---|---|
| | Model | Distal Shaft | Distal Jacket | (B) | (C) |
| No. | Name | Total Length | Length | Tip Length | Diameter |
| 1 | PBA52 | 100 mm ± 10% | 95 mm ± 10% | 5 mm ± 10% | 2.5 mm ± 10% |
| 2 | PBA72 | 80 mm ± 10% | 75 mm ± 10% | | |
| 3 | PBA82 | 70 mm ± 10% | 65 mm ± 10% | | |
| 4 | PBA54 | 100 mm ± 10% | 95 mm ± 10% | | |
| 5 | PBA74 | 80 mm ± 10% | 75 mm ± 10% | | |
| 6 | PBA84 | 70 mm ± 10% | 65 mm ± 10% | | |

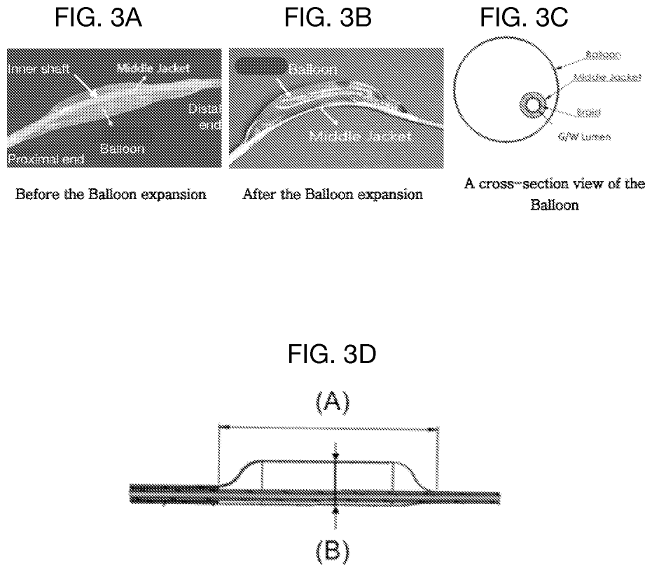

FIGS. 3A-3C show the details of the inflatable balloon 121. The balloon 121 is configured to be placed within the tricuspid valve to reduce the regurgitation. FIG. 3A shows the balloon 121 before expansion. FIG. 3B shows the expanded balloon 121. FIG. 3C shows a cross-section view of the balloon 121. Inside the balloon, there is an inner shaft 131 covered with the middle jacket as shown in FIG. 3B. The inner shaft 131 has a lumen for insertion of the guidewire. The balloon 121 is configured to expand at the tricuspid valve by injecting physiological saline to the pre-determined diameter.

As shown in FIGS. 3A-3B, the balloon has a distal end and a proximal end. The distal end of the balloon is mounted on the inner shaft, while the proximal end of the balloon is mounted on the distal portion of the proximal shaft 131. The braid on the inner shaft is for the control of the inner shaft strength and flexibility. The middle jacket of the inner shaft is for the support of the balloon. FIG. 3D shows the pre-defined dimensions of the balloon 121 having various lengths and diameters depending on the patient's anatomical structure (Table B).

TABLE B

| | | | (B) | | | |
|---|---|---|---|---|---|---|
| | | | After the Balloon expansion: | | | |
| | | | Balloon Diameter at Maximum Diameter Position | | | |
| | Model | (A) | Injection | Injection | Injection | Injection |
| No. | Name | Length | Volume 5 cc | Volume 10 cc | Volume 15 cc | Volume 20 cc |
| 1 | PBA52 | (50 ± 5) mm | (15 ± 3) mm | (19 ± 3) mm | (21 ± 3) mm | — |
| 2 | PBA72 | (70 ± 5) mm | (13 ± 3) mm | (17 ± 3) mm | (18 ± 3) mm | (21 ± 3) mm |
| 3 | PBA82 | (80 ± 5) mm | (12 ± 3) mm | (16 ± 3) mm | (18 ± 3) mm | (20 ± 3) mm |
| 4 | PBA54 | (50 ± 5) mm | (15 ± 3) mm | (19 ± 3) mm | (21 ± 3) mm | — |
| 5 | PBA74 | (70 ± 5) mm | (13 ± 3) mm | (17 ± 3) mm | (19 ± 3) mm | (21 ± 3) mm |
| 6 | PBA84 | (80 ± 5) mm | (12 ± 3) mm | (16 ± 3) mm | (18 ± 3) mm | (20 ± 3) mm |

FIGS. 4A-4D show the details of the proximal shaft 130 of the device 100. As shown in FIG. 4D, the proximal shaft 130 comprises an inner shaft 131 and an outer shaft 132. The outer shaft has a lumen for insertion of the inner shaft. The inner shaft has a lumen for insertion of the guidewire. Also, there is a gap between the inner and outer shafts for injection of the physiological saline for the balloon. The inner and outer shafts are configured to control the balloon (i.e., size and position rotation). Again, the outer shaft is a part through which the physiological saline is injected for the expansion of the balloon between the inner and outer shafts, on the outer side of the proximal shaft 130. The proximal jacket is a part made of harder Pebax material than the distal shaft, to allow further support to the distal shaft 110, on the inner side of the proximal shaft 130. The braid of the proximal shaft 130 is for the control of shaft strength and flexibility. FIG. 4E shows the pre-defined dimensions of the proximal shaft 130 having various lengths and diameters depending on the patient's anatomical structure (Table C).

TABLE C

| | | (A) | (B) |
|---|---|---|---|
| No. | Model Name | Length | Diameter |
| 1 | PBA52 | 550 mm ± 10% | 3.9 mm ± 10% |
| 2 | PBA72 | | |
| 3 | PBA82 | | |
| 4 | PBA54 | 565 mm ± 10% | |
| 5 | PBA74 | | |
| 6 | PBA84 | | |

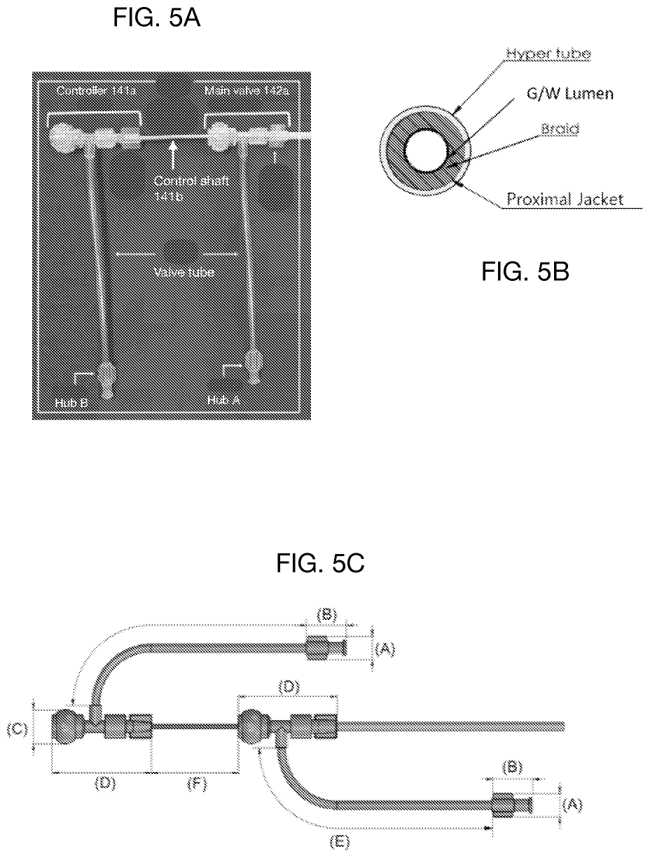

FIGS. 5A-5B show the details of the control segment 140. The controller 140 comprises a main valve 142a and a controller 141a. The main valve 142a further comprises a hub A. The controller 141a has a control shaft 141b and a hub B. The controller 141a is configured to bend or extend the distal shaft 110 through the connection to the proximal shaft 130, allowing the balloon 121 to be the accurately placed at the target position.

Hub A is configured to allow the injection of physiological saline or a contrast medium, with potential connection to a separate device for the balloon expansion and contraction. The main valve 142a is configured to be connected to the outer shaft 132 of the proximal shaft 130. Hub B is configured to act as an opening for the injection of physiological saline for shaft-internal flushing, etc.

The controller 141a is configured to be pushed or pulled to bend or extend the distal shaft 110 through the connection to the inner shaft 131 of the proximal shaft 130. Also, the control shaft 141b is configured to act as an opening to allow the passage of the guidewire to the distal shaft 110 and the proximal shaft 130, with an additional function to prevent hemorrhage. The valve tube is configured to have a passage through which physiological saline is injected through the hub. Finally, the control shaft 141b is configured to pass through the main valve lumen and configured to connect to the inner shaft 131 of the proximal shaft 130. FIG. 5C shows the pre-defined dimensions of the controller 140 having various lengths and diameters depending on the patient's anatomical structure (Table D).

TABLE D

| | | Hub A, Hub B | | Valve, Valve Body | | Valve Tube | Pusher Tube |
|---|---|---|---|---|---|---|---|
| | Model | (A) | (B) | (C) | (D) | (E) | (F) |
| No. | Name | Length | Length | Length | Length | Length | Length |
| 1 | PBA52 | 12 mm ± | 22 mm ± | 17 mm ± | 55 mm ± | 130 mm ± | 45 mm ± |
| 2 | PBA72 | 10% | 10% | 10% | 10% | 10% | 10% |
| 3 | PBA82 | | | | | | |
| 4 | PBA54 | | | | | | |
| 5 | PBA74 | | | | | | |
| 6 | PBA84 | | | | | | |

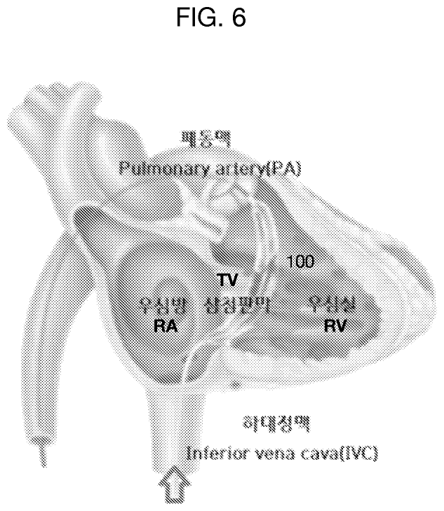

In another embodiment, as shown in FIG. 6, the device 100 is configured for size measurements to allow the selection of a suitable size of the implant for the patient, and an attached balloon with a changeable size. The device 100 is inserted along the previously inserted guide wire to position the balloon to span over the converged area of the tricuspid showing the regurgitation. A syringe with a diluted contrast medium (a separate product) is connected to Hub A, and the contrast medium is injected to the device, while the balloon 121 is being expanded to reach the target size. The diameter (size) of the balloon 121 is checked according to the injected dose of the contrast medium to determine the size of the affected area. In addition, with a transient (within 24 hour) insertion at the tricuspid, the improvement in TR can be monitored using the Echocardiogram and potential side effects after the surgical treatment can be predicted.

### Procedure

The procedures are performed in a suitable operation room (Cath Lab, etc.) to ensure adequate safety of the patient and allow Angiography/Fluoroscopy. First, a puncture is formed on the femoral vein and a sheath of 14 Fr or above is placed.

Using a catheter and a balloon catheter, a path is secured after confirming the lack of any obstruction or damage to the valve structure. The path is through the IVC and the tricuspid and towards the pulmonary artery.

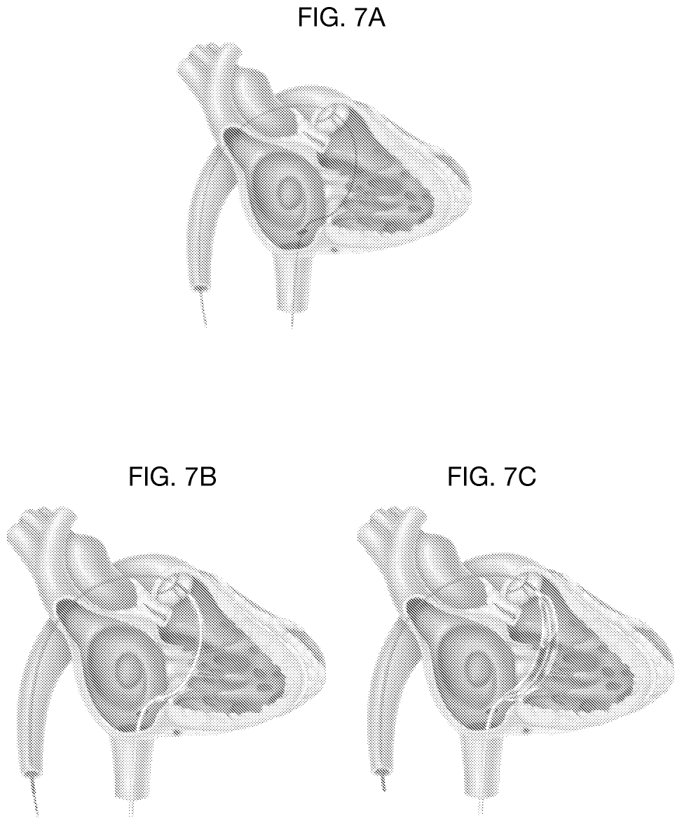

Along the catheter placed at the secured path, a guidewire is inserted. The guidewire is generally positioned deep towards the right lung as shown in FIG. 7A. In certain cases, the guidewire may be inserted towards the left lung based on the judgment of the surgeon.

Along the guidewire, the device 100 is inserted as shown in FIG. 7B. Using the TTE for monitoring, the balloon 121 is slowly expanded as shown in FIG. 7C. Checking the optimum position and the pattern of reduction in TR symptoms, the size of the TR area is measured.

After checking the area showing reduced TR symptoms and the size of the device 100, the guidewire and the device 100 is loaded, and after fixing the catheter on the femoral skin, the patient's state including therapeutic effects and side effects are monitored in an ICU or general/day ward that allows such monitoring.

In another embodiment, the loading time may be determined based on the judgment of the physician, while the device 100 should be removed before a day (24 h) has passed. Upon removal, the device 100 is contracted back to the initial state and pulled out along the guidewire in an opposite direction to the insertion. Following the removal of the device 100, the guidewire is pulled out of the body for removal.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of treating tricuspid valve regurgitation in a patient's heart, comprising:
having a transcatheter device comprising (i)-(iv) below;
(i) a main shaft;
(ii) a proximal portion comprising a proximal segment of the main shaft;
(iii) a distal tail comprising a distal portion of the main shaft;
(iv) a spacer body mounted on the main shaft and located between the proximal segment of the main shaft and the distal tail;
inserting the transcatheter device into a femoral vein;
advancing the transcatheter device through an inferior vena cava;
advancing the transcatheter device through a right atrium of the heart;
advancing the transcatheter device across a tricuspid valve and into a right ventricle of the heart;
advancing the transcatheter device towards a pulmonary artery;
advancing the distal tail into the pulmonary artery for a distance of at least 10 cm into the pulmonary artery; and
positioning the spacer body between leaflets of the tricuspid valve.

2. The method of claim 1, further comprising positioning the spacer body to abut against a supraventricular crest of the heart.

3. The method of claim 2, wherein the abutting against the supraventricular crest occurs at a location within the distal half of the spacer body.

4. The method of claim 2, wherein the tricuspid valve has a tricuspid annulus and there is an annular plane defined for the tricuspid annulus;
wherein the annular plane is along an x-axis of the tricuspid annulus and orthogonal to a Y-axis of the tricuspid annulus;
wherein the spacer body is positioned at an oblique angle relative to the annular plane.

5. The method of claim 2, wherein the main shaft comprises a lumen, and the method further comprises:
inserting a guidewire into the femoral vein;
advancing the guidewire through the inferior vena cava;
advancing the guidewire through the right atrium;
advancing the guidewire to traverse the tricuspid valve and into the right ventricle;
introducing the guidewire into the lumen of the shaft;
advancing the transcatheter device over the guidewire.

6. The method of claim 1, wherein the distal tail is advanced past a first branching point of the pulmonary artery.

7. The method of claim 6, wherein the distal tail is advanced past a second branching point of the pulmonary artery.

8. The method of claim 7, wherein the distal tail of the transcatheter device is advanced past a third branching point of the pulmonary artery.

9. The method of claim 1, wherein the distal tail of the transcatheter device is advanced a least 15 cm into the pulmonary artery.

10. The method of claim 1, wherein the spacer body has an opening through which blood flows through the spacer body.

11. The method of claim 1, wherein the pulmonary artery is a left-side pulmonary artery.

12. A transcatheter device comprising:
a main shaft having a distal tail, an inner shaft, and an outer shaft, the inner shaft having a lumen for insertion of a guidewire, the outer shaft having a lumen for insertion of the inner shaft;
an inflatable balloon mounted on the main shaft wherein a distal end of the balloon is mounted on the inner shaft and a proximal end of the balloon is mounted on a distal end of the outer shaft; and
a controller attached to a proximal end of the inner shaft in such a way the controller is configured to change the balloon's size and position.

13. The transcatheter device of claim 12, wherein the balloon has a non-linear shape.

14. The transcatheter device of claim 12, wherein the distal tail comprises at least a bend.

15. The transcatheter device of claim 12, wherein the distal tail comprises a proximal segment and a distal segment, wherein the distal segment has a thinner diameter than the proximal segment.

16. The transcatheter device of claim 12, wherein the balloon has a length in the range of range of 4-13 cm.
